(19) United States
(12) Patent Application Publication
(10) Pub. No.: US 2016/0095967 A1
(43) Pub. Date: Apr. 7, 2016

(54) HANDS FREE BRA KIT FOR REMOVING MILK FROM THE HUMAN BREAST

(71) Applicant: Richard Scott Weston, Encinitas, CA (US)

(72) Inventor: Richard Scott Weston, Encinitas, CA (US)

(21) Appl. No.: 14/503,592

(22) Filed: Oct. 1, 2014

(51) Int. Cl. A61M 1/06; A41C 3/04

(52) U.S. Cl. CPC A61M 1/062; A41C 3/04; A61M 2209/088

(57) ABSTRACT

The present invention provides hands free bra kit for removing milk from the human breast comprising an adjustable neck strap, a bottom strap, a top strap a pair of loops, a pair of tension balls and a reinforced area. The hands free bra kit is a fully portable and compact kit. The neck strap is provided around the neck, the top strap is provided around top of breast shed and the bottom strap is provided around the waist. The loop is configured for inserting horn of the breast and the tension ball holds an encircled material on the breast horn. The reinforced area can be a thicken portion or a grommet designed to take the weight of the bra kit. A method for placing hands free kit on breast and method for massaging breast to remove the milk completely is also disclosed.

### FIELD OF INVENTION

The embodiments herein generally relate to breast milk pumping. More specifically, the embodiments described herein relate to hands free bra kit for removing milk from the human breast.

### BACKGROUND OF THE INVENTION

Natural breastfeeding provides a bonding experience between a mother and a baby and is widely accepted as the best way to nurture an infant. Breastfeeding provides complete nutrition and it is also easy for the baby to digest. Breast milk has just the right amount of fat, sugar, water, and protein that is needed for baby's growth and development. Breastfeeding triggers biochemical reactions which allows for the enzymes, hormones, growth factors and immunologic substances to help create effective defense to infectious diseases for the infant. The breast milk also has long-chain polyunsaturated fatty acids which help with normal retinal and neural development and breastfeeding uses an average of 500 calories a day, which helps the mother to lose the weight after giving birth. The composition of breast milk changes depends on how long the baby nurses at each session, as well as on the age of the child. It also decreases allergies, decreases risk of diabetes and celiac disease and decreases the risk of sudden infant death syndrome for the infant. Benefits for the mother include decreases risk of breast cancer, decreases depression, and decreases risk of osteoporosis.

Babies should continue breastfeed for a year and for as long as is mutually desired by the mother and baby. Natural breast feeding has been widely accepted as the best way to nurture the infant. However, breastfeeding is not possible for working mothers and other women who are not able to be present at every feeding. Conversely, breast milk is good for babies and requires ongoing practice to sustain optimum lactation. For this purpose, expression of breast milk with a pump has been accepted as a practice for many years.

Although, milk obtained through breast pump devices provides convenience for infants but requires the mother to spend time pumping her breast for milk. Though, the breast pump devices takes mother time to use her hands to hold the breast pump is inconvenient and also requires the mother to delay accomplishing other tasks.

Further, the existing breast pump devices on the market have shown that these pumps cannot completely remove milk from the breast. Further, most existing pumps can remove only 40%-60% of the milk in the breast.

Therefore, there is a need in the art to develop a hands free bra kit for removing milk from the human breast to overcome the problem associated with the prior art. Further, there is a need for hands free bra kit for removing milk completely from the human breast.

### SUMMARY OF THE INVENTION

In view of the foregoing, an embodiment herein provides hands free bra kit for removing milk from the human breast comprising In an embodiment, the hands free bra kit comprising an adjustable neck strap, a bottom strap, a pair of loops, a pair of tension balls and a reinforced area. In another embodiment, the hands free bra kit comprising an adjustable neck strap, a bottom strap, a top strap a pair of loops, a pair of tension balls and a reinforced area. The hands free bra kit is a fully portable and compact kit and allows a mother to accomplish usual tasks. The neck strap is provided around the neck, which can be flexible or rigid, the top strap is provided around top of breast shed and the bottom strap is provided around the waist. Here, the loop is configured for inserting horn of the breast and the tension ball holds an encircled material on the breast horn. The reinforced area can be a thicken portion or a grommet designed to take the weight and wear of the bra kit.

According to an embodiment, the neck strap is rigid or flexible and combined with the tension ball, the loop, and the bottom strap. The bottom strap is an elastic waist strap and placed through the reinforced area. Further, a hole in the reinforced area is provided and limited in diameter, which helps to reduce the movement of the waist strap. Further, the reinforced area is made of a predetermined material and further, that area has been melted, or through any other reinforcing mechanism.

Since the bra kit is hands free, mothers can use their hand to massage their breast to remove the milk completely and collect in a container. According to an embodiment, the method for placing hands free bra kit on breast comprising the step of placing the bottom strap just below the breast area, wrapping the bottom strap around the waist, connecting both the end of bottom strap through a hole provided in a reinforced area, inserting the loops around the breast, fixing the suction pump on the nipple of breast, moving the loop on the top of the pump to keep/place the pump with the breast, and moving the tension ball towards the pump to tight and keep rigidly the pump with breast.

According to an embodiment, a method for massaging breast using the hands free kit comprising the step of placing the hands free kit on breast, applying suction of breast pump connected to the horns, allowing the pump to remove the milk as much as possible, massaging both the breast towards the nipple until the milk spray occurs, stop massaging when the milk flow slows down, and shaking the breast and repeating the messaging of breast until no milk present.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The detailed description is set forth with reference to the accompanying figures. In the figures the use of the same reference numbers in different figures indicates similar or identical items.

FIG. 1 illustrates front view of hands free bra kit for removing milk from the human breast, according to an embodiment herein;

FIG. 2 illustrates front view of hands free bra kit for removing milk from the human breast, according to another embodiment herein; and

FIG. 3 illustrates side view of hands free bra kit for removing milk from the human breast, according to an embodiment.

### DETAILED DESCRIPTION OF THE PREFERRED EMBODIMENTS

The embodiments herein and the various features and advantageous details thereof are explained more fully with reference to the non-limiting embodiments and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

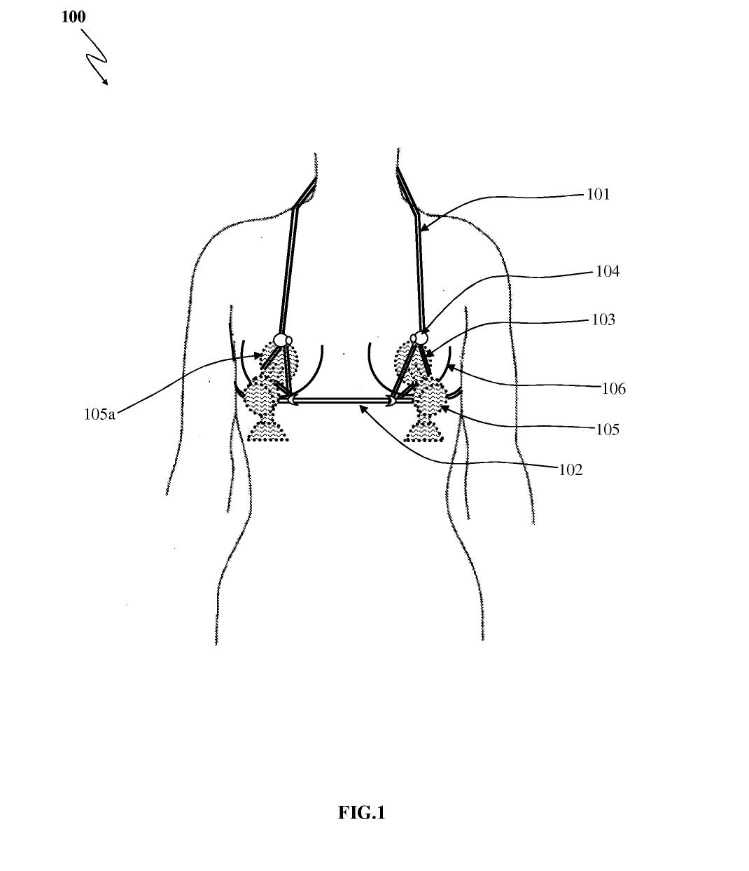

As mentioned above, there remains a need for hands free bra kit for removing milk from the human breast. The embodiments herein achieve this by providing hands free bra kit comprising at least an adjustable neck strap, a waist strap, a pair of loops, a pair of tension balls and a reinforced area. Referring now to the drawings, more particularly to FIGS. 1 through 3, where similar reference characters denote corresponding features consistently throughout the figures, there are shown preferred embodiments.

According to an embodiment, hands free bra kit is a fully portable and allows a mother to accomplish usual tasks. The hands free bra kit is very compact and straps present in the bra kit supports the mother in a comfortable manner.

According to an embodiment, the hands free bra kit uses pumping along with the massaging action of the breast is effective in delivering milk with increased milk output.

According to an embodiment, FIG. 1 illustrates front view 100 of hands free bra kit for removing milk from the human breast. The hands free bra kit comprising an adjustable neck strap 101, a bottom strap 102, a pair of loops 103, a pair of tension balls 104 and a reinforced area [not shown].

According to an embodiment, the bottom strap 102 is an elastic waist strap and placed through the reinforced area, and a hole provided in the reinforced area is limited in diameter to reduce the movement of the bottom strap 102. The bottom strap 102 is placed just below the breast 106 area, and the bottom strap 102 can be wrapped around the waist and both the end of the bottom strap can be connected together with the help of reinforced area. The reinforced area can be a thicken portion or a grommet designed to take the weight of the bra kit. The reinforcing area is configured to allow the bottom strap to pass through. Further, the reinforcing area also provides resistant to keep the loop/neckstrap in place so it does not wander around.

In an embodiment, the adjustable neck strap 101 is connected to the loop 103 via the tension balls 104. The neck strap 101 is wearable around the neck and the length of the neck strap 101 can be adjusted as the user requirement. The neck strap 101 is rigid or flexible and combined with the loop 103 in order to keep two pumps 105 rigidly with breast 106 of the user.

According to an embodiment, the tension ball 104 can move along the length of neck strap 101. The bottom of the loop 103 is connected to the bottom strap 102 and is placed under the breast 100, and the size/area of the loop 103 can be adjusted with the movement of the tension ball 104, according to an embodiment. The tension ball 104 can be moved upwards or downwards towards the pump 105, thus the size/area of the loop 103 can be varied. Accordingly, the loop 103 is configured for inserting the horn of the breast, and the tension ball 104 can be moved towards the pump in order to keep the pump 105 intact with the breast 106 of user, and the tension ball 104 enables to hold encircled material on the breast horn 105a.

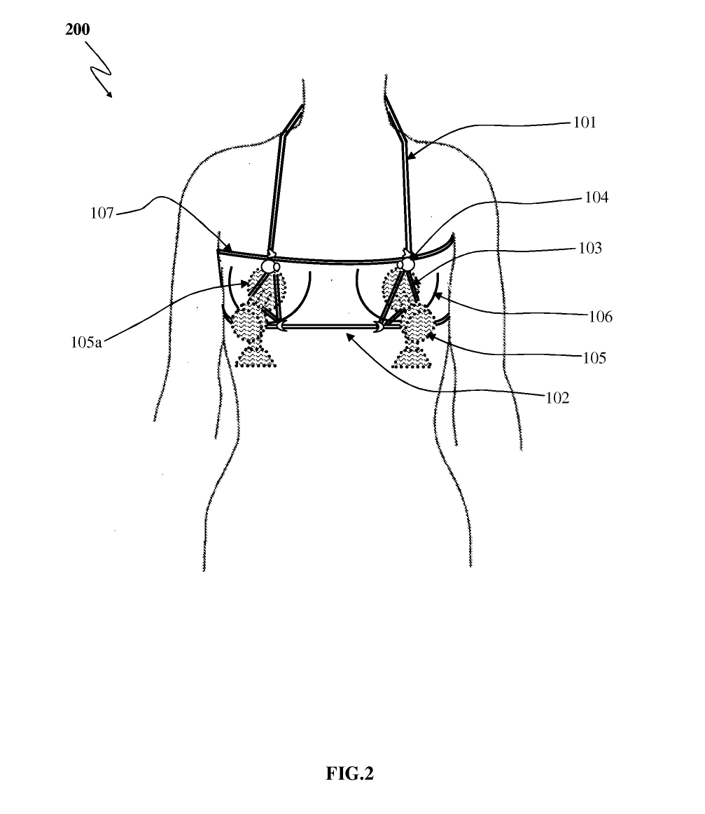

FIG. 2 illustrates front view 200 of hands free bra kit for removing milk from the human breast, according to another embodiment. The hands free bra kit comprising an adjustable neck strap 101, a bottom strap 102, a pair of loops 103, a pair of tension balls 104, a top strap 107 and a reinforced area [not shown].

In an embodiment, the top strap 107 can be provided around top of breast shed and the top strap 107 is connected with the tension balls 104 in order to keep the loop 103 and hold with the pump 105. The configuration and functionality of other elements such as the adjustable neck strap 101, the bottom strap 102, the pair of loops 103, the pair of tension balls 104 work similar as first embodiment.

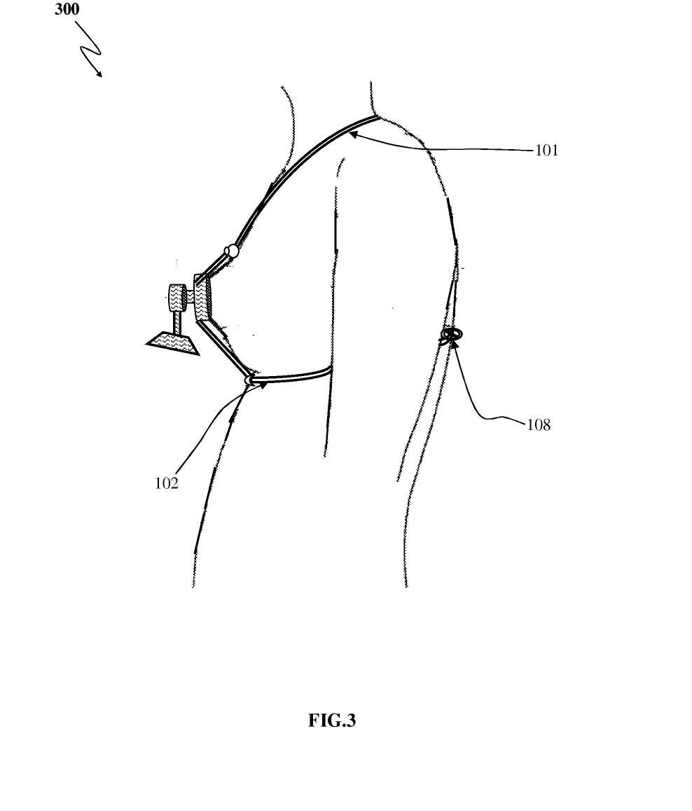

FIG. 3 illustrates side view of hands free bra kit for removing milk from the human breast, according to an embodiment. The bottom strap 102 placed through the reinforced area 108, wherein a hole is provided and diameter of the hole is limited to reduce the movement of the bottom strap 102.

Since the bra kit is hands free, mothers can use their hand to massage their breast 106 to remove the milk completely and collect in a container. According to an embodiment, the method for placing hands free bra kit on breast comprising the step of placing the bottom strap 102 just below the breast 106 area, wrapping the bottom strap 102 around the waist, connecting both the end of bottom strap through a hole provided in a reinforced area 108, inserting the loops 103 around the breast 106, fixing the suction pump 105 on the nipple of breast 106, moving the loop 103 on the top of the pump 105 to keep the pump 105 with the breast 106, and moving the tension ball 104 towards the pump 105 to tight and keep rigidly the pump 105 with breast 106.

According to an embodiment, a method for massaging breast using the hands free kit comprising the step of placing the hands free kit on breast, applying suction of breast pump connected to the breast, allowing the pump to remove the milk as much as possible, massaging both the breast towards the nipple until the milk spray occurs, stop massaging when the milk flow slows down, and shaking the breast and repeating the messaging of breast until no milk present.

According an embodiment, the hands free bra kit can be used for pumping the milk in the breast and present massaging method supports for increasing milk output. Thus, the hands free bra kit along with the present massaging technique enables for collecting milk completely from the human breast.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A hands free bra kit for removing milk from the human breast comprising:
at least an adjustable neck strap; a bottom strap; a pair of loops; a pair of tension balls; and a reinforced area.

2. The hands free bra kit of claim 1, wherein said neck strap is provided around the neck for supporting the pump, wherein said bottom strap is provided around the waist.

3. The hands free bra kit of claim 1, wherein said neck strap is rigid or flexible and combined with said tension balls, said loops and said bottom strap.

4. The hands free bra kit of claim 1, wherein said loop is configured for inserting horn of the breast.

5. The hands free bra kit of claim 1, wherein said tension ball can move along the length of the neck strap which enables to hold an encircled material on the breast horn.

6. The hands free bra kit of claim 1, wherein said reinforced area is a thicken portion to take the weight of the bra kit.

7. The hands free bra kit of claim 1, wherein said bottom strap is made of elastic material and said bottom strap is placed through the reinforced area.

8. A hands free bra kit for removing milk from the human breast comprising:
at least an adjustable neck strap; a bottom strap; a top strap, a pair of loops; a pair of tension balls; and a reinforced area.

9. The hands free bra kit of claim 8, wherein said top strap is provided around top of breast shed.

10. The hands free bra kit of claim 8, wherein said neck strap is rigid and combined with said tension balls, said top strap, said loops and said bottom strap.

11. A method for placing hands free bra kit on breast comprising the step of
placing the bottom strap just below the breast area;
wrapping the bottom strap around the waist;
connecting both the end of bottom strap through a hole provided in a reinforced area;
inserting the loops around the breast, fixing the suction pump on the nipple of breast;
moving the loop on the top of the pump to keep/place the pump with the breast; and
moving the tension ball towards the pump to tight and keep rigidly the pump with breast.

12. A method for massaging breast using a hands free kit comprising the step of
placing the hands free kit on breast;
applying suction of breast pump connected to the breast;
allowing the pump to remove the milk as much as possible;
massaging the breast towards the nipple until the milk spray occurs;
stop massaging when the milk flow slows down; and
shaking the breast and repeating the messaging of breast until no milk present
